(19) United States
(12) Patent Application Publication
(10) Pub. No.: US 2026/0232939 A1
(43) Pub. Date: Aug. 13, 2026

(54) MEDICAL NASAL RESPIRATORY DEVICE AND USES THEREOF

(71) Applicant: Basim Abdelfattah Ali Elgazzar, Shebin Elkom, (EG)

(72) Inventor: Basim Abdelfattah Ali Elgazzar, Shebin Elkom, (EG)

(21) Appl. No.: 18/703,355

(22) PCT Filed: Jul. 31, 2022
(86) PCT No.: PCT/EG2022/000029

§ 371 Date: Oct. 21, 2024

(30) Foreign Application Priority Data
Oct. 25, 2021 (EG) 2021101691

(51) Int. Cl. A61M 16/06; A61M 16/00; A61M 16/20

(52) U.S. Cl. CPC A61M 16/0672; A61M 16/0003; A61M 16/208

(57) ABSTRACT

The present invention relates to a medical nasal device for administrating a continuous stabilized flow oxygen supply to an infant or a toddler comprising (a) a horizontal hollow tube has two nasal prongs; (b) a cuff/balloon mounted on the free end of the prongs; (c) a small filling tube; d) Safety pressure relief valve with a pilot ballon attached to the end of the filling tube and acts as a one way valve with pressure relief and a detachable syringe is connected to it, wherein the mentioned device is fixed easily without injuries or any damage wherein the prongs do not touch neither the septum nor the lateral aspects and the horizontal tube does not touch the columella (lower part of the septum). It provides adequate sealing without any leak.

### FIELD OF THE INVENTION

The present invention generally relates to a nasal adapter device and method for using it in CPAP and mechanical non-invasive ventilation in the field of intensive care and home-based treatment of obstructive sleep apnea. In particular, the invention relates to a nasal respiratory device and its uses.

### BACKGROUND OF THE INVENTION

Non-invasive ventilation interfaces include nasal prongs, nasal masks and oronasal masks. All forms of masks used for non-invasive ventilation and continuous positive airway pressure (CPAP) (whether at home for obstructive sleep apnea or in hospital) do not provide adequate sealing, their tight application causes nasal injuries to an infant, a toddler or an adult and are difficult to be fixed in position.

Any cpap therapy needs:
1—Careful positioning.
2—Vigilant nursing care.

US20080047559 relates to a ventilation apparatus that involves a manifold and 2 nasal engagement knobs with surrounding air chambers that are inflated with air around the knobs. This device does not provide adequate fixation in place as the air chambers around the nasal knobs extend along the whole length of the knobs so on its inflation, the upper part of each knob will lie in the nasal cavity, the lower part of each knob will remain outside the nares which will lead to its easy extrusion and displacement with minimal movement. Also, if pushed upwards into the nasal cavity to the maximum it will bring the manifold in contact with the columella and will act as a pressure point on the nasal columella causing pressure necrosis. The air chambers are filled by a pump that is actuated by the patient's hand which makes it liable for over inflation and pressure necrosis of the nasal mucosa (no precise volume of injected air) or under inflation which causes leak (no precise specific volume of air or fluid injected). It also does not allow injection of fluid, only air. Also, the pump volume is fixed while the size of the nasal cavities differs from one patient to another with different cuff volumes required to seal the nasal cavities of different patients (does not offer individual adjustability). Besides, the size of the pump adds to the weight and size of the whole device.

U.S. Pat. No. 5,269,296 relates to a device where the cuffs are not mounted on the top of the prongs, they surround a portion of the prongs leading to extension of a portion of the prongs beyond the upper border of the cuffs inside the nasal cavity (inside the nares) which will cause nasal irritation and increase the possibility of mucosal injury given the triangular anatomy of the nasal cavity.

Cuff apertures that communicate the cannula lumen with the cuffs add to the complexity of the design plus it is an inaccurate and inefficient way to inflate and maintain the inflation of the cuffs especially during the inspiratory efforts of the patient. The cuffs' volume will vary during inspiration and expiration disrupting the seal. It depends on the direction of air current through the aperture and the pressure gradient between the cuffs and the cannula lumen (which are in turn communicating with the patient's airways). The volume of the cuffs is small compared to the patient's airways where the pressure varies throughout the respiratory cycle so it may either underfill the cuffs thus disrupting the seal or overfill the cuffs causing an increase in the pressure in the cuffs with subsequent injury, ischemia to the nasal mucosa and pain.

In the other embodiment, the inflation tubes are connected to a source of inflation pressure such as a hand operated compression bulb or an electrical compression pump, this is an inaccurate and inefficient way of inflating the cuffs. Firstly, maintenance of inflation will not be achieved as there is no check valve (the air will leave the cuffs back to the pump once the squeeze is released). Only a release valve is provided for cuff deflation. Secondly, a hand operated compression bulb or an electrical compression pump will not inject a precise accurate volume of air (a small precise amount of air is all that is needed to inflate the cuffs). It may inject a high volume causing severe distension of the cuffs with subsequent pain and annoyance to the patient and a high pressure in the cuffs or a low volume with defective seal (does not account for individual variability). It also does not allow injection of fluid, only air.

The air harness (and its air filling mechanism, rigid hose, buttress segments, hook and loop fastener system . . . ) adds to the weight of the device and to the complexity of the design and is cumbersome to use by the patient (surrounds the head and may give a sensation of claustrophobia). An air harness is needed in this design to retain the device in position to compensate for the defective fixation in position caused by the fact that the cuff inflation will not be maintained. If the cuff inflation could be maintained, then adequate fixation in position will be achieved thus omitting the need for a harness. Since the harness will be filled with air from the CPAP air supply, a higher pressure will be needed to supply pressure to the patient.

The steel and metal components add to the weight and complexity of the mask and the structure called “cannula support” adds to the complexity of the design.

US20200360637 relates to a complex design that is designed to deliver a high flow gas to the patient. It has cuffs on the prongs. The overall structure is designed so that cuffs inflate during inspiration and deflate during expiration or during delivery of high flow gas (HFG). In expiration there is no side stream flow to the cuffs, so they deflate, and the interface seal is released so the patient is able to passively expire, with gas flow exhaling around the prongs. The inflation deflation movement of the cuffs during inspiration and expiration respectively is not ideal for CPAP delivery. (i.e. the volume of the cuffs is not fixed during inspiratory expiratory movement). In the alternative design the cuffs deflate through a vent device (2262) when high flow gas is initiated. When high flow gas is initiated, it operates a valve (2340) that unblocks the vent device (thus deflating the cuffs). Besides adding to the complexity, it also leads to a continuous inflation deflation movement of the cuffs (the volume of the cuffs is not fixed throughout the respiratory cycle of inspiration and expiration).

The cuffs are filled by a side stream that is caused by hand squeezing of a bag which is not an accurate way of filling the cuffs with a specific volume of air and my cause either overinflation with subsequent pressure necrosis of the nasal mucosa or underinflation and absent sealing.

The deflation movement is not precise (it cannot be guaranteed that the exact volume that inflated the cuffs will get out in expiration leading to complete deflation) and can be incomplete causing gradual accumulation of air in the cuffs with the subsequent bag squeezing leading to increased pressure inside them above the safe limit with subsequent pressure necrosis of the nasal mucosa.

The cuffs do not surround the top part of the prongs. They surround the lower part and may be in fluid communication with each other, which causes defective fixation in position as the upper part of the cuffs will lie in the nasal cavity while the lower part will remain outside so the prongs can be extruded with movement of the head. The continuous inflation and deflation of the cuffs will cause positional instability and will help the extrusion of the prongs. Part of the prongs will extend beyond the upper border of the cuffs in the nasal cavity causing mucosal irritation and injury.

U.S. Pat. No. 7,331,348B1 A nasal CPAP cannula seal that includes a strip of flexible material, between about 3 to 10 cm in width and between about 5 to 15 cm in length. The strip defines a pair of substantially round nostril apertures, each aperture having a diameter of between 2 mm and 4.5 mm and being spaced apart by from 1.5 mm to 3 mm. Also, the strip further defines a set of cuts extending outwardly from each nostril aperture. It will cause skin irritation and leak will occur between the prongs and the strip.

The inventor found that the problem and side effects of any CPAP or non-invasive ventilation interface in the market include:
1—Pressure points: skin damage and breakdown, nasal septal, columellal and lateral wall injuries. Called also hospital acquired pressure injuries (HAPI),
2—Lack of positional stability: frequently the prongs get out of the nostrils especially with infants.
3—Leakage which causes the physician to either:
Reseat the prongs more firmly in the nares which causes septal breakdown
Resizing of the prongs which causes pressure breakdown on the exterior aspect of the nares.
Escalation to more invasive measures e.g. intubation.
4—Using a head gear to augment fixation in position. This is hard in multiple removals and applications.
5—Even in the prior designs that utilize cuffs to create a seal and avoid leakage, the cuffs were inaccurately positioned on the prongs with improper inflation methods of the cuffs ultimately leading to poor fixation in position, inappropriate inflation with either underinflation and leakage or overinflation with pain, irritation and mucosal damage.

As is apparent from the above explanations of the prior art, the disadvantage of all known nasal is that they do not seal 100%, there is no secure fixation in position and there is a risk of nasal mucosal injury.

### SUMMARY OF THE INVENTION

The present invention relates to a medical nasal device and method for administering a continuous stabilized flow of oxygen supply to an infant, to a toddler or to an adult; or for administering non-invasive ventilation, comprising (a) A horizontal hollow tube (attached on one side to the expiratory limb & the other side is attached to the inspiratory limb) and two nasal prongs; (b) 2 cuffs/balloons surrounding the upper end (the free end) of the prongs so that no part of the prongs extend beyond the upper border of the cuffs; (c) a small filling tube; (d) Safety one-way valve (with pressure release to avoid excess pressure in the cuffs) with an attached pilot ballon. (e) A detachable syringe can be connected to the valve, to inject a specific and precise volume of air or fluid into the cuffs). The device is fixed easily without injuries or any damage: When the device is applied to the patient the prongs are pushed into the nasal cavity up to the lower border of the cuffs. The horizontal tube does not touch the columella (lower part of the septum), the prongs do not extend beyond the upper border of the cuffs. The device provides adequate sealing without any leak and is easily fixed in position.

The advantages of the present invention are as following:
1—Easy fixation in position. The cuffs will lie completely inside the nasal cavities as they surround the free end of the prongs only. Once in situ with inflation of the cuffs it will be secured in place.
2—Avoiding injuries. There will always be a space between the barrel (horizontal tube) and the nasal septum (no touching the septum or the columella). The prongs do not touch either the septum or the lateral aspects of the nares and do not extend beyond the upper border of the cuffs when inside the nasal cavity. The cuff will not cause any damage as long as the pressure inside the cuff does not exceed the pressure inside the capillaries of the nasal mucosa (extra pressure is released through the valve).
3—Adequate sealing without any leak. Cuff inflation is maintained (due to the presence of the check valve) and the volume of the cuffs is fixed (similar during inspiration and expiration).
4—Easy application and removal without the cumbersome head gears being worn by the patient.
5—Can be used for CPAP or for non-invasive ventilation.
6—Cuff inflation is precise and gradual; this allows easy adjustability for individual needs and the syringe is detached after inflation which makes the device small and light weighed.

### BRIEF DESCRIPTION OF THE FIGURES

The foregoing and other features and aspects of the present technology can be better understood from the following description of embodiments and as illustrated in the accompanying drawings. The accompanying drawings, which are incorporated herein and form a part of the specification, further serves to illustrate the principles of the present technology. The drawings are not necessarily to scale.

FIG. 1 schematically illustrates an embodiment of the medical nasal device including all its components.

FIG. 2 schematically illustrates a section in the device.

### DETAILED DESCRIPTION OF A PREFERRED EMBODIMENT

In one aspect, the invention provides a medical nasal device for administering a continuous stabilized flow of oxygen supply to an infant, a toddler or an adult (CPAP or non-invasive ventilation), comprising; (a) A horizontal hollow tube (1) has two ends “an inspiratory end (2) & an expiratory end (3) connected to the inspiratory and expiratory limbs respectively” and two nasal prongs (4), wherein the mentioned prongs (4) protrude perpendicularly from the horizontal tube (1); (b) A cuff/balloon (5), which is mounted on each nasal prong and surrounds the free end of the prong (facing the patients nose) with no part of the prong extending beyond the cuff, a carboxymethylcelluclose (CMC) coating could be added to the cuff or a local anesthetic or lubricant gel could be applied to it before application of the device to the patient; (c) A filling tube (7), which bifurcates from one side into two terminal filling tubes (6) and attached on the other (free) side to a safety valve (8) to fill each cuff with air or fluid such that the mentioned device provides no leakage, no loss of pressure and avoids cartilaginous and septal nasal injuries; (d) Safety one-way valve with pressure release and a pilot ballon (8) attached to the free end of the filling tube (7) and acts as a one way valve and a detachable air/fluid syringe is connected to it. The small filling tube (7) and the 2 terminal filling tubes (6) may be embedded in whole or part in the wall of the horizontal tube (1) and the prongs (4).

The horizontal hollow tube is attached on one side to the inspiratory limb and on the other side to the expiratory limb to be used as a CPAP or mechanical ventilator and after application of the device to the patient, this horizontal tube does not contact the columella.

The small filling tube is attached on the free side to a safety valve and a detachable syringe is connected to the valve to fill the cuffs gradually till adequate sealing is achieved; allowing adjustability for individual needs.

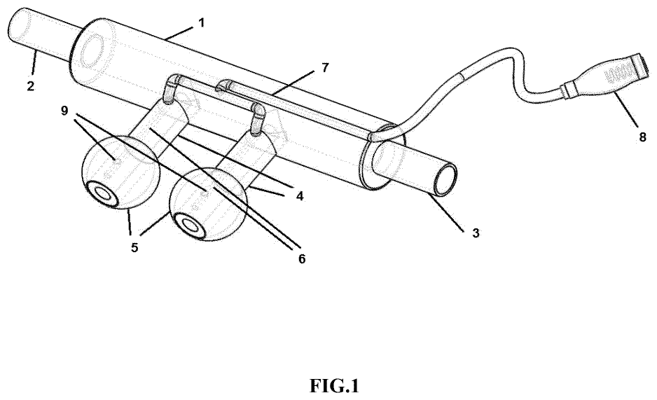

FIG. 1 shows the medical nasal device of the present invention comprising:
A horizontal hollow tube (1);
An inspiratory limb (2);
An expiratory limb (3);
Two nasal prongs (4);
Cuffs/balloons (5);
Terminal filling tubes (6);
A filling tube (7);
Safety valve with pressure release and a pilot ballon (8);
Openings of the filling tubes inside the cuffs (9).

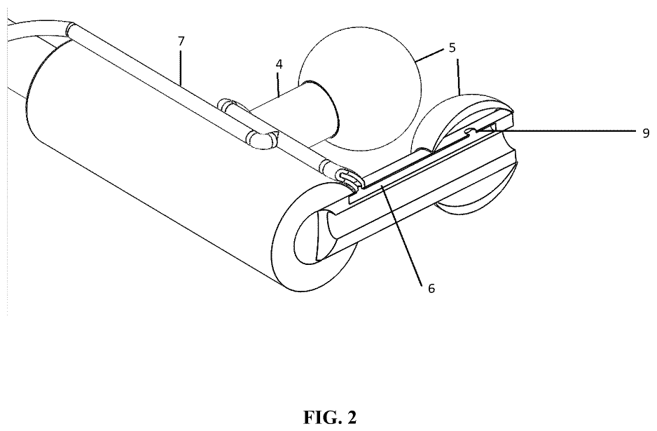

FIG. 2 shows a cross section in one structure exposing a prong, a filling tube and a cuff)
Nasal prong (4);
Cuff/balloon (5);
Internal filling tube in cut section (6);
Small filling tube (7);
Opening of the filling tube inside the cuff (9).

In a further aspect, the invention provides a method for administering a continuous stabilized flow of oxygen supply by using the device, wherein the mentioned method comprising the following steps:
(a) The prongs (4) are inserted into the nostrils with the cuffs (5) deflated and advanced into the nasal cavities until the cuffs lie completely inside the nasal cavities. Local anesthetic could be applied to the cuffs before insertion, or they could be immersed in water if they were CMC coated. (b) The cuffs (5) are inflated securing the prongs in position to avoid any leak, and then the device is connected to the gas supply wherein a mouth strap may be added to ensure further sealing if necessary. This horizontal tube (1) does not contact the columella. To remove the device, the air is withdrawn out of the cuffs by actuating the valve using the syringe and the device is pulled out of the nose thereafter.

In an embodiment, the cuffs (5) of the present invention may assume any shape: pear shaped, pyramidal, tapering, rounded, fusiform . . . etc.

The device may be made of silicone, PVC or polyurethane (PU) . . . etc. The cuffs are inflated with air and rarely other substances: saline, alkalinized lidocaine, nitrous oxide . . . etc.

The cuffs' compliance could vary but high compliance (high volume low pressure) is preferred and the pressure in the cuffs should not exceed the capillary pressure in the nasal mucosa to avoid ischemia with extra air/fluid being released by the valve.

What we claim is:

1- A medical nasal device of variable sizes for administering CPAP or mechanical ventilation to an infant, a toddler or an adult, comprising: (a) A horizontal hollow tube with two ends (an inspiratory end & an expiratory end) connected to the inspiratory and expiratory limbs of a gas supply machine respectively and two nasal prongs made of a strong material protruding perpendicularly from the horizontal tube; (b) 2 cuffs/balloons (right and left), each of them is mounted on the free end of its respective nasal prong; (c) A filling tube, which bifurcates into two terminal filling tubes to fill each cuff with air or fluid; d) Check valve with pressure release and an optional pilot ballon attached to the filling tube from one side and fits a detachable syringe on the other side to inject a specific and precise volume of air or fluid into the filling tube such that when the device is applied to the patient's nasal cavity all of the cuffs, terminal tubes, filling tube, the optional pilot ballon and check valve maintain an equal adequate pressure ensuring proper fitting, adequate nasal cavity sealing, and no collateral tissue damage but not collapsing the prongs.

2- A medical nasal device according to claim 1 wherein the 2 cuffs are mounted on the free end of the prongs with optional carboxymethylcelluclose or other soothing coating and when adequately inflated ensures sealing of the nasal cavity.

3- A medical nasal device according to claim 1 wherein the 2 cuffs can assume any suitable shape: horizontal, pyramidal, pear shaped . . . etc to fix the device in position when adequately inflated.

4- A medical nasal device according to claim 1 wherein the 2 cuffs surround only the free end of the prongs so that no part of the prongs protrudes into the nasal cavity.

5- A medical nasal device according to claim 1 wherein the filling tube and the 2 terminal tubes intermediate between the cuffs on one side and the check valve with the optional pilot ballon on the other side.

6- A medical nasal device according to claim 1 wherein the check valve with pressure relief (with an optional pilot ballon) is attached to the filling tube on one side and fits a detachable syringe on the other side to inject precise volume of air or fluid to the cuffs and prevents its spontaneous deflation to achieve adequate sealing without overinflation and pressure injury or underinflation with disrupted seal.

7- A medical nasal device according to claim 1 wherein the pressure inside the cuffs is high enough to inflate the cuffs so that it ensures adequate sealing, yet it is less than the capillary pressure of the nasal mucosa.

8- A medical nasal device according to claim 1 wherein the device has various sizes to accommodate infants, toddlers and adults of different sizes, races and facial measures.

9- A method for administering a continuous stabilized flow of oxygen supply by using the device aforementioned, wherein the mentioned method comprising the following steps:
(a) The prongs are inserted into the nostrils with the cuffs deflated until the cuffs completely lie inside the nasal cavities.
(b) The cuffs are inflated securing the prongs in position to avoid any leak, and then the device is connected to the gas supply wherein an optional mouth strap may be added to ensure further sealing; and on removal the air/fluid is drained from the cuffs by actuating the valve using a syringe then the device is pulled out.

10- The method of claim 9 for administering a continuous stabilized flow of oxygen supply by using the device of claim 1, wherein the cuffs are inserted into the nasal cavities completely with optional application of a lubricant gel.

11- The method of claim 9 for administering a continuous stabilized flow of oxygen supply by using the device of claim 1, wherein the pressure inside the cuffs is controlled by the air/fluid injected using the syringe.

12- The method of claim 9 for administering a continuous stabilized flow of oxygen supply by using the device of claim 1, wherein the pressure in claim 11 is high enough to achieve sealing but does exceed the capillary pressure of the nasal mucosa to avoid pressure necrosis and tissue damage.
